(19) United States
(12) Patent Application Publication
(10) Pub. No.: US 2020/0005929 A1
(43) Pub. Date: Jan. 2, 2020

(54) Psychotherapy Triage Method

(71) Applicant: IESO DIGITAL HEALTH LIMITED, Cambridge (GB)

(72) Inventors: Mihai Valentin Tablan, Cambridge (GB); Ronan Cummins, Cambridge (GB); Andrew Blackwell, Cambridge (GB)

(73) Assignee: IESO DIGITAL HEALTH LIMITED, Cambridge (GB)

(21) Appl. No.: 16/486,467

(22) PCT Filed: Mar. 1, 2018
(86) PCT No.: PCT/EP2018/055080

§ 371 Date: Aug. 15, 2019

(30) Foreign Application Priority Data
Mar. 1, 2017 (GB) 1703343.2
Sep. 21, 2017 (GB) 1715251.3
Jan. 12, 2018 (GB) 1800557.9

(51) Int. Cl. G16H 20/70; G16H 50/20; G06N 20/00

(52) U.S. Cl. CPC G16H 20/70; G06N 20/00; G16H 50/20

(57) ABSTRACT

A computer-based psychotherapy triage method comprising: obtaining text data relating to a patient at an initial stage of a therapy process; using at least a first part of a deep learning model to obtain a representation of at least the text data; using at least a second part of the deep learning model, and an input thereto formed using the representation, to obtain an output predicting a characteristic of a condition of the patient and/or of the therapy process; and causing the system to take one or more actions relating to the therapy process, wherein the one or more actions are selected based on the output; wherein the deep learning model is trained using a training set comprising, for a plurality of other patients, text data relating to the other patient at an initial stage of a therapy process and a result of a determination of the characteristic.

### FIELD

The present application relates among other things to a method for use by a computer-based system for providing (psycho)therapy.

### BACKGROUND

A computer-based system for providing therapy is described in WO 2016/071660 A1 (which is hereby incorporated by reference). Among other things, the system enables patients and therapists to exchange messages, particularly text-based messages, during sessions of therapy. This application relates to certain technical improvements to such a system.

Common mental health disorders including depression and anxiety are characterized by intense emotional distress, which affects social and occupational functioning. About one in four adults worldwide suffer from a mental health problem in any given year. In the US, mental disorders are associated with estimated direct health system costs of $201 billion per year, growing at a rate of 6% per year, faster than the gross domestic product growth rate of 4% per year. Combined with annual loss of earnings of $193 billion, the estimated total mental health cost is at almost $400 billion per year. In the UK mental health disorders are associated with service costs of £22.5 billion per year and annual loss of earnings of £26.1 billion.

Traditional models of the provision of care for individuals with common mental health disorders rely on face-to-face sessions of therapy, for example cognitive behavioral therapy (CBT), delivered in person between a therapist and a patient. Whilst this standard care approach may be effective for some patients, it has significant drawbacks in terms of, amongst other things, convenience to the patient, cost of the provision, accessibility of the therapist to the patient outside booked session times, ongoing assessment of the patient's progress or improvement between sessions, and supervision of the therapist.

Online therapy, including internet-enabled cognitive behavioral therapy (IECBT), offers significant advantages over standard care. Internet-enabled cognitive behavioral therapy (IECBT) is a type of high-intensity online therapy used within an Improving Access to Psychological Therapies (IAPT) program. Within IAPT using IECBT, patients are offered weekly one-to-one sessions with an accredited therapist, similar to face-to-face programs, whilst also retaining the advantages of text-based online therapy provision including convenience, accessibility, increased disclosure and shorter waiting times. The improvement rate for patients treated with IECBT is significantly higher than for severity-matched patients treated with standard care.

One element of both standard and IECBT care is that a patient needs to be assessed before they can commence treatment. For example, the most likely presenting condition (diagnosis) for the patient, and the severity of that patient's condition, must be ascertained so that appropriate care that is likely to be effective for the patient (e.g. a particular treatment protocol, and/or an appropriate amount of therapy/number of therapy sessions) can be offered. This initial assessment may currently be conducted by the therapist using information from e.g. standardized questionnaires, for example patient health questionnaire (PHQ-9) scores and/or general anxiety disorder (GAD-7) scores, in addition to other information gathered from the patient. In part, the correct diagnosis for a patient relies on the therapist's experience in interpreting the results of the questionnaires and the other information gathered from the patient. One drawback of the current assessment methodology is that an incorrect initial assessment by a therapist may result in the provision of inappropriate care for that patient (e.g. the wrong treatment protocol being adhered to), which could result in a lack of improvement or even a deterioration in the patient's condition, in addition to a waste of the patient's and therapist's time and other resources with associated costs, even if the incorrect initial assessment is later corrected.

Further information useful at the start of the therapy process may include what the likelihood is of the patient not engaging with the therapy process or dropping out of therapy early. In particular, there is a chance that some patients will not engage with the therapy process even before an initial assessment by the therapist has been carried out; these patients are entirely lost to the therapy process and will not therefore benefit from it. Other patients may drop out of therapy before it is completed, i.e. before they have gained maximum therapeutic benefit from it, this represents a cost to the patient as they may not therefore improve and/or recover. Early drop out is also wasteful in terms of, for example, the cost of therapy already delivered, the time and other resources already committed by the patient and/or the therapist, and the fact that if therapy is subsequently re-engaged with by the patient they may require an additional amount of therapy beyond what might have been sufficient previously. If it can be reliably determined that a patient is at risk of not engaging or dropping out of the therapy process, interventions can be deployed to that patient in order to mitigate the chance of non-engagement/drop-out occurring, and reduce the associated costs. Current methods of determining the chance of a patient dropping out rely on the experience of the therapist, and the patient reliably self-reporting their own engagement level, both of which are at least in part subjective, and cannot by definition identify those patients who do not engage at all with the therapy process beyond initial contact.

For these reasons, a new approach is required to improve, augment or assist with initial assessment of a psychotherapy patient.

### SUMMARY

According to a first aspect of the present invention, there is provided:
a method for use by a computer-based system for providing psychotherapy, the method comprising:
obtaining, via a user interface of the system, text data relating to a patient at an initial stage of a therapy process;
using at least a first part of a deep learning model to obtain a representation of at least the text data;
using at least a second part of the deep learning model, and an input thereto formed using the representation, to obtain an output predicting a characteristic of a condition of the patient and/or of the therapy process; and
causing the system to take one or more actions relating to the therapy process, wherein the one or more actions are selected based on the output;
wherein the deep learning model is trained using a training set comprising, for each of a plurality of other patients, text data relating to the other patients at an initial stage of a therapy process and a result of a determination of the characteristic.

A representation of at least the text data may be for example a tensor representation, a higher order tensor representation, an at least third-order tensor representation, a matrix representation, or a vector representation. The representation of at least the text data may be a tensor representation, more specifically it may be a numeric tensor representation or a dense (numeric) tensor representation. The representation of at least the text data is sometimes referred to as a final representation. Therefore the representation of at least the text data may be a tensor representation, for example a matrix or higher order representation. It will be understood that the order of the tensor will be appropriate to the complexity of the information it represents.

Dense representations (tensors) are preferred in deep learning methods for at least two reasons. Firstly, they are more compact than sparse representations; this is because sparse representations are very simple, but occupy a considerably larger amount of space to represent the same amount of information. Secondly, dense representations are more expressive than sparse representations, as they are capable of encoding the degree of relatedness of different input values. For example, when representing categorical data similar values may receive representations that are numerically close, whereas dissimilar values may receive representations that are more numerically distant. For example, in relation to text data, synonyms may be represented by numerically-close vectors, whilst unrelated words may be represented by numerically-distant vectors.

Thus, the method may increase the effectiveness and/or the efficiency of the system by using deep learning processes to predict e.g. a characteristic of a condition of the patient and by taking one or more actions relating to the therapy process accordingly. The one or more actions may be selected based on a predicted severity of a condition and so the method may be referred to as digital triage.

The text data may comprise free-form text (e.g. answers provided by the patient to open-ended questions). Compared to other types of data relating to the patient, free-form text may be readily obtained and may provide a richer source of information for predicting the characteristic. Free-form text contains a large amount of information, this may therefore be too much information for a therapist, even an experienced therapist, to use effectively when predicting a characteristic of a patient (e.g. their presenting condition or likelihood of drop-out). In contrast with this, the large amount of data obtained from free-form text may be beneficial to the method, as it may permit more effective training of the model, and/or more accurate prediction of the characteristic.

The method may comprise obtaining further data (e.g. personal data, medical data, etc.) relating to the patient. The method may comprise obtaining the representation by at least: obtaining an intermediate representation of the text data; obtaining a further intermediate representation of the further data; and joining the intermediate representations. Thus, a maximal amount of available data may be used for predicting the characteristic. Therefore all the data available may be made available to the deep learning model of the method; furthermore, during performance of the method the deep learning model may learn which elements of data are useful for the prediction of a characteristic of a condition of the patient and/or of the therapy process, and which elements of data appear to be irrelevant. Using a maximal amount of available data (text data and further data) may therefore increase the accuracy of the method. For example, all the available information relating to a patient, including patient demographic data, medical history data, and the free text supplied by the patient, may be represented, and joined into a dense numeric representation, typically as a higher order tensor.

An intermediate representation of the text data may be for example a tensor representation, a higher order tensor representation, an at least third-order tensor representation, a matrix representation, or a vector representation. The intermediate representation of the text data may be a tensor representation, more particularly it may be a numeric tensor representation or a dense (numeric) tensor representation. An intermediate representation of the text data may also be described as features, as a set of features, or as a representation. Therefore the intermediate representation may be a tensor representation, for example a matrix representation. It will be understood that the order of the tensor will be appropriate to the complexity of the information it represents.

A further intermediate representation of the further data may be for example a tensor representation, a higher order tensor representation, an at least third-order tensor representation, a matrix representation, a vector representation, or a scalar representation. The further intermediate representation of the further data may be a tensor representation, more specifically it may be a numeric tensor representation or a dense (numeric) tensor representation. The further data may be patient data. A further intermediate representation of the further data may also be described as a further representation or as a representation. Therefore the further intermediate representation may be a tensor representation, for example a scalar representation or a vector representation. For example, numeric values, such as a patient's age, may be represented as a scalar. Categorical values, such as a patient's gender, may be represented (embedded) as vectors. It will be understood that other types of input may also be converted into dense tensors, of orders appropriate to the complexity of the information.

Obtaining the representation may comprise pre-processing an intermediate representation of at least the text data, wherein the pre-processing may comprise, for example, normalising. Such a normalisation may make the representation (considerably) more suitable for use with the second part of the deep learning model. Therefore, obtaining the representation may comprise normalising an intermediate representation of at least the text data. Normalising is typically used to improve the numeric stability of the learning process, helping it to converge faster.

The first part of the deep learning model may be pre-trained using in-domain text (e.g. text relevant to psychotherapy). Thus, among other things, the representation may represent more meaningful features of the text data. Pre-training the first part of the deep learning model using in-domain text may be advantageous, because it may control for word semantics that differ slightly from the general usage of the word(s). This ensures the system starts with a suitable representation of word meanings, which reduces the amount of training required. Pre-training using in-domain text may comprise using text that is all in-domain; alternatively pre-training using in-domain text may comprise initially pre-training using general text and then further pre-training using in-domain text.

The second part of the deep learning model may perform classification or regression. The method may perform a plurality of instances of classification and/or regression to obtain a plurality of outputs. The output or outputs may comprise:
a most likely condition at the initial stage (e.g. a presenting condition/problem);
a likelihood score for each of a set of possible conditions at the initial stage;
a predicted severity of a condition at the initial stage;
a predicted amount of therapy (a treatment amount) required;
a likelihood of non-engagement and/or drop-out by the patient; and/or
one of a plurality of therapy options that is most likely to be beneficial.

Training the model to obtain multiple (i.e. a plurality of) outputs may be beneficial, as it may encourage it to discover general representations of the data (text data, further patient data), rather than representations that are narrowly focused on a single task (output). General representations, that are proven useful for multiple tasks, are more likely to be accurate than representations that produce a single output. Therefore training the model to obtain a plurality of outputs may have a synergistic effect. Training the model to obtain a plurality of outputs may otherwise be known as multi-task learning, and may be considered a form of regularization of the model.

The one or more actions may comprise allocating the patient to one of a plurality of therapists. The allocation may be based at least in part on a predicted characteristic and on data describing performance of the therapist in relation to the predicted characteristic. Thus, the method may match patients with therapists who are likely to provide more effective and/or efficient psychotherapy to the patient. Alternatively or additionally, the allocation may be based on a predicted severity of a condition at the initial stage and on data describing experience of the therapist (e.g. in relation to the condition). Patients with more severe conditions are allocated to therapists with more experience. Thus, the method may use therapist resources in an optimal way. The allocation may also be based on further data (e.g. data relating to availability, etc.).

As explained in WO 2016/071660 A1, the system may enable patients and (allocated) therapists to make appointments for sessions, exchange messages during the sessions, etc.

The one or more actions may comprise selecting at least one of a plurality of therapy plans based on the output and providing, via a user interface of the system, an indication of the selected at least one therapy plan to the therapist. Thus, the method may automatically suggest appropriate therapy plans. This may be advantageous as the selection of the therapy plan may be less subjective than a selection determined by a therapist; therefore the patient is less likely to be allocated to a therapy plan of lower potential benefit to that patient; the misallocation of a therapy plan is associated with increased cost to the patient and/or the therapy provider or service.

The method may further include making the system assist the therapist in following a selected therapy plan. This may be referred to as therapist assistance.

The one or more actions may comprise, in response to the likelihood of non-engagement and/or drop-out by the patient meeting a predetermined criterion, deploying at least one of a plurality of interventions, wherein the at least one intervention is predicted or known to increase engagement. It is advantageous to be able to predict which patients are at higher risk of non-engagement and/or drop out and therefore to differentially deploy at least one intervention with those patients, because this may therefore reduce the overall cost to the therapy provider/service of providing intervention(s), whilst at the same time achieving a reduction in non-engagement and/or drop out occurrence amongst patients (which represents a cost to the patient of non or reduced improvement or recovery). It is advantageous to be able to predict which patients are at higher risk of non-engagement and/or drop out before it occurs, rather than reacting to drop-out after it has happened, because intervention(s) deployed in advance of drop out may be more effective in increasing engagement, and therefore less likely to result in a cost to the patient. In addition, the ability to predict likelihood of non-engagement/drop-out may present a further economic benefit to the therapy provider or therapy service in pay-for-performance therapy models.

The one or more actions may comprise, in response to a predicted severity of a condition being below a predetermined criterion or threshold, initiating a therapy process that involves providing information to the patient via the system. In particular, the system may initiate a therapy process that does not directly (or indirectly) involve a therapist. Thus, the method may avoid unnecessary use of therapists. The predetermined criterion or threshold may be a (predetermined) severity criterion or severity threshold. The avoidance of unnecessary use of therapists may be advantageous to both therapy providers/services and patients; for example therapy services may not incur unnecessary associated costs (e.g. the cost of paying therapists to provide unnecessary therapy; the further cost of reducing the availability of therapists who could otherwise be treating patients with more severe conditions), whereas patients benefit from receiving a therapy plan more appropriate to their needs, which may be beneficial in terms of e.g. convenience and/or speed of delivery.

The method may comprise selecting a subset of a set of information based on the output and providing, via a user interface of the system, the selected information to the therapist and/or to the patient. The information may include documents, questionnaires, etc. The information may be provided at appropriate times, e.g. before or during particular sessions. Thus, the method may help the therapist and the patient during the therapy process.

The method may comprise: subsequently determining the characteristic of a condition of the patient and/or of the therapy process; and selectively updating the training set and/or re-training the deep learning model. Thus, the accuracy and reliability of the predictions may be continually increased. The subsequent determination of the characteristic of a condition of the patient and/or of the therapy process may be a determination made by a therapist following interaction with the patient in the course of therapy. These subsequent determinations may be used to further train the system such that its accuracy may improve over time or with increasing amounts of data.

According to a further aspect of the present invention, there is provided a computer program for performing the method.

According to a further aspect of the present invention, there is provided a non-transitory computer-readable medium comprising a computer program according to the preceding claim.

According to a further aspect of the present invention, there is provided a computer-based system configured to perform the method.

The system may comprise:
one or more servers;
one or more communications networks; and
a plurality of devices configured to communicate with the one or more servers via the one or more communications networks.

Each server/device may comprise at least one processor and at least one memory comprising computer program code, the at least one memory and the computer program code configured to, with the at least one processor, cause the server/device to perform at least part of the method.

According to a further aspect of the present invention, there is provided
a method comprising:
vectorising a first text data relating to a patient at an initial stage of a therapy process to produce a plurality of first text data tensors;
extracting a plurality of features that represent the first text data from the plurality of first text data tensors using a first portion of a deep learning model;
analysing a representation, based on at least the plurality of features that represent the first text data, with a classification/regression portion of the deep learning model, thereby producing an output correlated to at least one characteristic of a condition of the patient and/or a related therapy process; and
categorising the patient based on the output;

wherein the deep learning model is trained using at least second text data from other patients at an initial stage of a therapy process and a corresponding characteristic of a condition of the other patients.

The method may further comprise vectorising patient data relating to the patient to produce a plurality of patient data tensors. The representation analysed by the classification/regression portion of the deep learning model of the method may further be based on the plurality of patient data tensors.

The deep learning model of the method may be further trained using in-domain text.

The classification/regression portion of the deep learning model of the method may perform a classification process on the representation. The at least one characteristic of a condition of the patient and/or the related therapy process may comprise a most likely condition of the patient at the initial stage and/or a likelihood score for each of a set of possible conditions at the initial stage.

The categorising of the patient based on the output of the method may comprise allocating the patient to one of a plurality of therapists.

The classification/regression portion of the deep learning model of the method may perform a regression process on the representation. The at least one characteristic of a condition of the patient and/or the related therapy process may comprise a predicted severity of the condition at the initial stage.

The predicted severity of the condition at the initial stage as determined by the method may be below a threshold. In that case, categorising the patient based on the output may comprise initiating a therapy process that initially does not directly involve a therapist. The threshold of the method may be a severity threshold.

The predicted severity of the condition at the initial stage as determined by the method may be above a threshold. In that case categorising the patient based on the output may comprise initiating a therapy process with an experienced therapist. The threshold of the method may be a severity threshold.

The classification/regression portion of the deep learning model of the method may perform a regression process on the representation. The output correlated to at least one characteristic of a condition of the patient and/or the related therapy process may comprise a predicted amount of therapy required and/or one of a plurality of therapy options that is most likely to be beneficial.

The classification/regression portion of the deep learning model of the method may perform a regression process on the representation. The output correlated to at least one characteristic of a condition of the patient and/or the related therapy process may comprise a likelihood of non-engagement and/or drop-out by the patient.

The categorising of the patient in accordance with the method of the invention may involve deploying at least one of a plurality of interventions. The at least one intervention may be predicted or known to increase engagement.

### BRIEF DESCRIPTION OF THE DRAWINGS

Certain embodiments of the present invention will now be described, by way of example, with reference to the accompanying drawings, in which:

FIG. 1 illustrates a system for providing psychotherapy.

FIG. 2a illustrates a device which may form part of the system of FIG. 1.

FIG. 2b illustrates a server which may form part of the system of FIG. 1.

FIG. 3 illustrates a method which may be carried out by the system of FIG. 1.

FIG. 4 illustrates a data flow diagram associated with the method of FIG. 3.

FIG. 5 illustrates an example of a display which may be provided by the method of FIG. 3.

FIGS. 6a-d illustrate different action steps of the method of FIG. 3.

FIG. 7 illustrates another method which may be carried out by the system of FIG. 1.

FIG. 8 illustrates the performance of the system with respect to the prediction of drop out/non-presentation of patients.

### DETAILED DESCRIPTION OF THE CERTAIN EMBODIMENTS

Computer-Based System

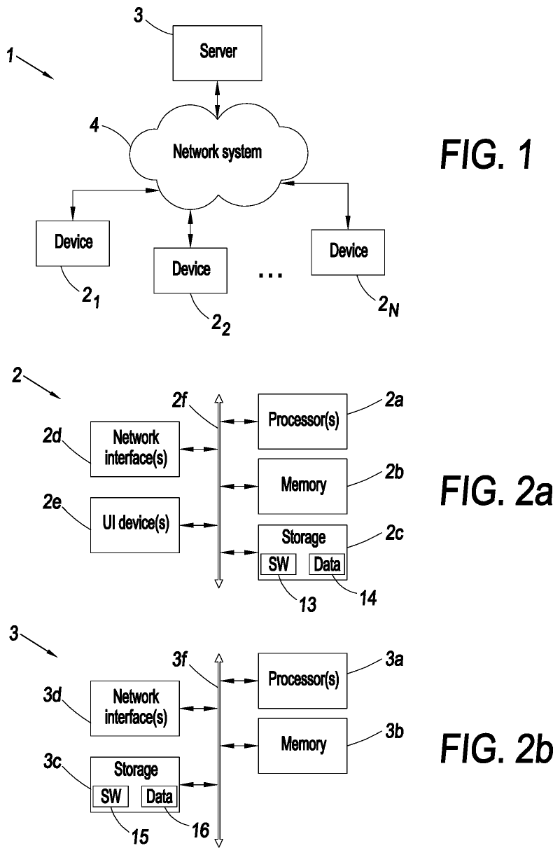

Referring to FIG. 1, a computer-based system 1 for providing psychotherapy includes a plurality of devices 2₁ 2_{N} connectable to a server 3 via a network system 4.

The system 1 preferably enables therapists and patients to use devices 2 to exchange text-based messages during sessions of therapy.

Each device 2 may be a mobile device, such as a laptop, tablet, smartphone, wearable device, etc. Each device 2 may be a (nominally) non-mobile device, such as desktop computer, etc. Each device 2 may be of any suitable type, such as a ubiquitous computing device, etc.

Referring to FIG. 2a, a (typical) device 2 includes one or more processors 2a, memory 2b, storage 2c, one or more network interfaces 2d, and one or more user interface (UI) devices 2e. The one or more processors 2a communicate with other elements of the device 2 via one or more buses 2f, either directly or via one or more interfaces (not shown). The memory 2b includes volatile memory such as dynamic random-access memory. Among other things, the volatile memory is used by the one or more processors 2a for temporary data storage, e.g. when controlling the operation of other elements of the device 2 or when moving data between elements of the device 2. The memory 2b includes non-volatile memory such as flash memory. Among other things, the non-volatile memory may store a basic input/output system (BIOS). The storage 2c includes e.g. solid-state storage and/or one or more hard disk drives. The storage 2c stores computer-readable instructions (SW) 13. The computer-readable instructions 13 include system software and application software. The application software preferably includes a web browser software application (hereinafter referred to simply as a web browser) among other things. The storage 2c also stores data 14 for use by the device 2. The one or more network interfaces 2d communicate with one or more types of network, for example an Ethernet network, a wireless local area network, a mobile/cellular data network, etc. The one or more user interface devices 2e preferably include a display and may include other output devices such as loudspeakers. The one or more user interface devices 2e preferably include a keyboard, pointing device (e.g. mouse) and/or a touchscreen, and may include other input device such as microphones, sensors, etc. Hence the device 2 is able to provide a user interface for e.g. a patient or therapist.

Referring to FIG. 2b, a (typical) server 3 may include one or more processors 3a, memory 3b, storage 3c, one or more network interfaces 3d, and one or more buses 3f. The elements of the server 3 are similar to the corresponding elements of the abovedescribed device 2. The storage 3c stores computer-readable instructions (SW) 15 (including system software and application software) and data 16 associated with the server 3. The application software preferably includes a web server among other things.

The server 3 may be different from the abovedescribed server 3. For example, the server 3 may correspond to a virtual machine, a part of a cloud computing system, a computer cluster, etc.

Referring again to FIG. 1, the network system 4 preferably includes a plurality of networks, including one or more local area networks (e.g. Ethernet networks, Wi-Fi networks), one or more mobile/cellular data networks (e.g. 2^{nd}, 3^{rd}, 4^{th} generation networks) and the Internet. Each device 2 is connectable to the server 3 via at least a part of the network system 4. Hence each device 2 is able to send and receive data (e.g. data constituting messages) to and from the server 3.

Method

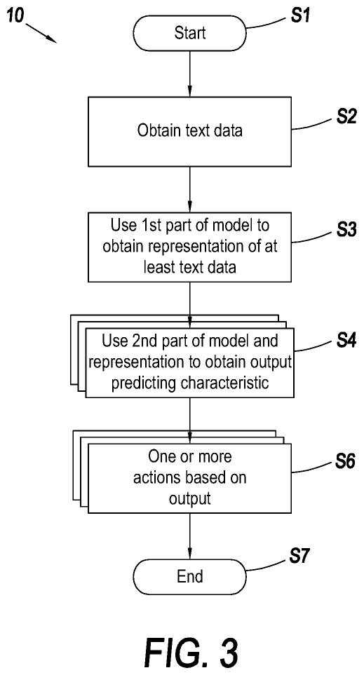

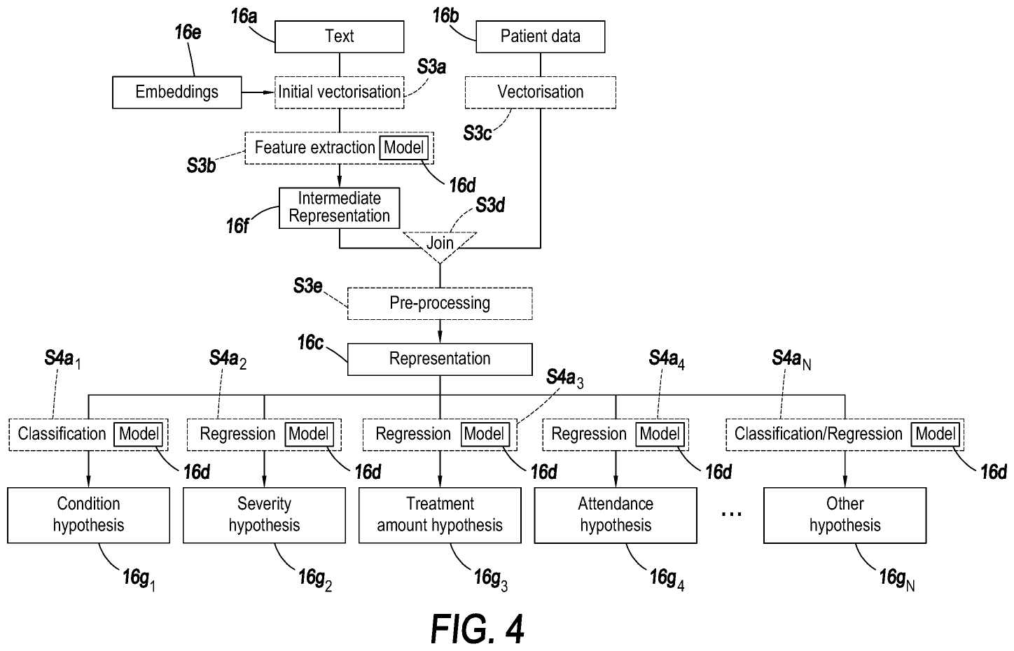

Referring to FIGS. 3 and 4, the system 1 may perform a method 10 comprising several steps S1-S7.

Training and Prediction Phases

As will become apparent, some steps, particularly the third and fourth steps S3, S4, may be performed either as part of a training phase or as part of a prediction phase.

The third and fourth steps S3, S4, each involve parts of a deep learning model. Such a model typically has model inputs, model parameters and model outputs.

Training data (hereinafter referred to as a training set) is used during the training phase. In some examples, the training set includes multiple instances of e.g. human-labelled data. During the training phase, the instances of data is provided as model inputs, and the model parameters are adjusted (i.e. the model is constructed) such that the model outputs optimally predict the corresponding labels. All of the data in the training set is used collectively to construct the model.

During the prediction phase, an instance of unlabelled data is inputted to the constructed model which outputs a corresponding prediction of the label.

First Step of the Method

Referring in particular to FIG. 3, at a first step S1, the method 10 starts. The first step S1 may involve a user of a device 2 (hereinafter referred to as a patient) causing the device 2 to establish a communications session with the server 3.

The device 2 and/or the server 3 preferably enable the patient to register, to identify and authenticate themselves, etc.

Typically, the device 2 and the server 3 communicate with one another during a communications session and run particular application software (including a web browser, a web server, further application software at the server 3, etc.).

In this way, the device 2 and the server 3 provide a user interface (hereinafter referred to as a patient interface) enabling the patient to interact with the system 1.

In a similar way, a device 2 and the server 3 provide a user interface (hereinafter referred to as a therapist interface) enabling a therapist to interact with the system 1.

Second Step of the Method

At a second step S2, the system 1 obtains certain text 16a (FIG. 4). The text 16a relates to the patient. The text 16a is preferably provided by the patient. The text 16a preferably includes free(form) text, i.e. any text may be provided by the patient. The text 16a may be English or any other language. The text 16a may be provided as part of a self-assessment questionnaire (hereinafter referred to as simply the questionnaire) completed by the patient using the patient interface. The questionnaire preferably includes open-ended questions. The questionnaire preferably asks the patient to explain their reasons for seeking help. For example, the questionnaire may include questions such as:
Describe the main problem you are bringing to therapy
When did the problem start—how long have you been feeling this way?
Can you describe a recent example of this problem?

The text 16a may be obtained in any suitable way. For example, the text 16a may be provided by the patient by typing, speaking (in which speech recognition is performed), etc. The text 16a need not be provided directly by the patient.

The method may also involve obtaining further data 16b (FIG. 4) relating to the patient (this further data is hereinafter referred to as patient data). The patient data 16b may include personal data such as age, gender, etc., medical data such as medication use, drugs/alcohol misuse, etc., and so forth. The patient data 16b may be provided by the patient using the patient interface or may be obtained in any other suitable way.

Third Step of the Method

At a third step S3, a (final) representation 16c of at least the text 16a (e.g. the text 16a and optionally the patient data 16b) is obtained.

As will be explained in more detail below, this involves using deep learning processes which may be referred to as a first part or first portion of a deep learning model 16d (FIG. 4).

Referring in particular to FIG. 4, at a first sub-step S3a, the text 16a is (initially) vectorised.

Generally, vectorisation means a process of converting data of an arbitrary type into a sequence of numbers, i.e. a vector. Vectorisation as used herein may also mean the production of higher order numeric structures such as matrices, or more generally, tensors of any order.

In this instance, the vectorisation involves replacing each word or short phrase with an associated embedding (an embedding associated with a word is hereinafter sometimes referred to as a word embedding).

A word embedding is a numeric representation of a word. Word embeddings associate words with positions in a highly dimensional vectorial space which is constructed such that similar or related words are positioned close to one another (according to some suitable distance metric).

Embeddings may be used to transform text (which is a sequence of words) into a sequence of vectors. Embeddings may be used to represent text in a form that is particularly suitable for use in deep learning processes. The vectorisation may allow deep learning processes to reason in semantic space rather than words space. Put differently, decisions may be made based on meanings of words rather than on the words themselves. Moreover, words that have not been seen before (i.e. words that have not appeared in the training set) may still be understood, provided that words with similar or related meanings have been seen.

In some examples, a set of embeddings 16e used to vectorise the text 16a may start as random numeric values and then be adjusted during the training phase.

However, preferably, the set of embeddings 16e is pre-trained based on statistics of word occurrences in corpuses of (unlabelled) text. Such pre-training may allow subsequent training during the training phase to converge faster and/or to reach a higher accuracy.

The pre-training preferably involves analysing large quantities of in-domain text, i.e. text related to psychotherapy. The in-domain text may include transcripts of therapy sessions conducted via the system 1 and/or text obtained from other sources such as public internet forums relating to mental health, blog posts, etc. The pre-training is intended to produce numerical representations associated with individual words or short phrases that exhibit desirable behaviour. An example of desirable behaviour is that vectorial distances between representations reflect semantic similarity or relatedness. The pre-training may involve the use of Word2vec (see T. Mikolov, K. Chen, G. Corrado and J. Dean, “Efficient Estimation of Word Representations in Vector Space,” arXiv preprint arXiv:1301.3781, vol. abs/1301.3781, 2013), GloVe (see J. Pennington, R. Socher and C. D. Manning, “GloVe: Global Vectors for Word Representation,” in Empirical Methods in Natural Language Processing (EMNLP), 2014) or other algorithms.

At a second sub-step S3b, a set of features 16f representing the text 16a (as a whole) is extracted. This set of features is hereinafter sometimes referred to as a representation, an intermediate representation, or features. The set of features 16f representing the text 16a may be a tensor representation, a matrix representation, a vector representation, or a numeric (scalar) representation. Where the set of features 16f representing the text 16a is a tensor representation, it may be a numeric tensor representation or a dense numeric tensor representation. Therefore the intermediate representation 16f may be a tensor, for example a vector representation.

Extracting the set of features (intermediate representation) 16f involves using the first part or portion of the deep learning model 16d. The first part of the deep learning model may include a single layer or multiple stacked layers. The layers may be of various types, such as convolutional neural network layers (see Y. LeCun, L. Bottou, Y. Bengio and P. Haffner, “Gradient-based learning applied to document recognition,” Proceedings of the IEEE, vol. 86, no. 11, p. 2278, 1998), recursive or recurrent neural network layers, long short-term memory layers (see S. Hochreiter and J. Schmidhuber, “Long short-term memory,” Neural computation, vol. 9, no. 8, p. 1735, 1997), fully connected neural network layers, drop-out layers, and various nonlinearities such as sigmoid, tanh, ReLU, etc.

A deep neural network (DNN) refers to an artificial neural network endowed with complex structure. A convolutional neural network (CNN) is a type of DNN developed for object recognition in images. Recent research suggests that CNNs can also be applied to text, where they can spot linguistic indicators. CNNs ignore most text structure and are only sensitive to very local dependencies. A recurrent neural network (RNN) is a type of DNN that is sensitive to text structure. RNNs are particularly effective at encoding the semantics of short- and medium-length text snippets (up to a sentence). RNNs do not currently work very well on whole documents, although recent developments (e.g. RNNs with attention) attempt to address this issue.

An advantage of the abovedescribed deep learning processes is that they automatically produce a (substantially) optimised feature representation during the training phase. In contrast, a popular feature representation in classical natural language processing (NLP) is n-grams. Each word (1-gram), pair of words (2-gram), triplet of words (3-gram), etc. constitutes a feature. A piece of text is represented by indicating the number of occurrences of each of the features (most of which will be zero). This approach may cause problems due to the very large potential feature space (given a 10,000 word vocabulary, there are 1 trillion potential trigrams) and data sparseness (most potential features are never observed in training data). In contrast, the deep learning processes may produce a very compact representation. Therefore the deep learning processes of the method may produce a very compact intermediate representation, very compact further intermediate representation, and/or very compact final representation. The representations produced by the deep learning processes of the method may further be numeric tensor representations, more particularly dense (numeric) tensor representations, meaning that most values are not zero. This is advantageous because dense representations are capable of encoding the degree of relatedness of different input values. For example, when representing categorical data similar values may receive representations that are numerically close, whereas dissimilar values may receive representations that are more numerically distant. For example, in relation to text data, synonyms may be represented by numerically-close vectors, whilst unrelated words may be represented by numerically-distant vectors.

Individual words and phrases are first represented by embeddings (dense vectors), which have a constant size regardless of the size of the vocabulary.

Whole pieces of text are then represented by so-called thought vectors, i.e. fixed-length numeric vectors that are derived by composing the embeddings. The way in which the embeddings are composed is determined during the training phase such that the resulting representation is most useful for distinguishing between the various outcome labels. While the details are different, the same is conceptually true when using either CNNs or RNNs to form the (intermediate) representation 16f. The other types of layer that are described above may be used to fine-tune the representation 16f.

Alternatively, following the representation of each word as a dense vector (embedding), all the words in the input text are chained together, forming a matrix wherein the representation of each word is a row. When multiple text extents need to be modelled, e.g. the separate responses to multiple questions, these may be grouped together into a higher order tensor. Alternatively, the multiple text extents may be simply appended, producing a taller matrix, with more rows.

At an optional third sub-step S3c, patient data 16b is vectorised. This vectorisation is performed using processes that are suitable for the type of data being encoded. For example, numeric data may be left as is or may be quantised, e.g. by allocating it to predetermined buckets. Categorical data may be converted to a dummy representation and then into multiple binary values.

Like the text data 16a, the vectorisation of the patient data 16b may use numeric embeddings (not shown) that are initialised at random and then adjusted during the training phase. This may allow the artificial neural network to automatically derive representations (not shown) that are most useful in the decision process, and encode similarities and differences between input values in a way that is more relevant to the process being modelled. A representation of the patient data 16b may be referred to as a further representation, or as a further intermediate representation. The further intermediate representation may be a tensor, for example a vector representation.

At a fourth sub-step S3d, the representation 16f of the text data 16a and the representation of the patient data 16b are joined. Thereby the intermediate representation, also known as the (set of) features, and the further intermediate representation, are joined.

In examples in which the patient data 16b is not used, the third and fourth sub-steps S3c, S3d are not performed and the (intermediate) representation 16f of the text data 16a is used directly in the subsequent sub-step S3e.

At a fifth sub-step S3e, suitably, the (intermediate) representation 16f, or joined (intermediate) representation 16f and (further intermediate) representation from step S3d, are pre-processed to form suitable inputs for the subsequent classification and/or regression processes. The pre-processing may involve various processes for the normalisation of feature values, such as standardization, whitening transformation, the application of drop-out at training time, etc.

Thus, a representation 16c (hereinafter referred to as a final representation) is obtained. This final representation may be a tensor, for example a higher-order tensor or a matrix.

Fourth Step of the Method

Referring particularly to FIG. 3, at a fourth step S4, at least one classification/regression process is used to obtain an output predicting a characteristic of a condition of the patient and/or of the therapy process. The output may also be referred to as a hypothesis. The output may represent a correlation with at least one characteristic of a condition of the patient and/or of a related therapy process, as generated by at least one classification/regression process of the method.

Referring particularly to FIG. 4, several classification and/or regression processes S4a₁-S4a_{N} are preferably used to obtain several such outputs 16g₁-16g_{N}.

A classification process is a machine learning process that associates categorical labels with input data. A regression process is a machine learning process that associates numerical labels/values with input data.

The one or more classification/regression processes S4a may be referred to as the second part of the deep learning model 16d. The one or more classification/regression processes S4a may also be referred to as the classification/regression portion of the deep learning model. The classification/regression portion of the deep learning model may be used to analyse the representation based on at least the plurality of features that represent the first text data. Analysis will be understood to mean the performance of classification and/or regression.

Where there are several classification and/or regression processes S4a₁-S4a_{N}, the same final representation 16c is used as an input to all of the classification and/or regression processes S4a₁-S4a_{N}. Sharing the final representation 16c in this way acts as a further regularization element and nudges the training toward an accurate and unbiased representation.

Various outputs 16g of interest may be obtained, e.g.:
a most likely presenting condition (a diagnosis), ‘Condition hypothesis’, 16g₁;
a predicted severity of the presenting condition, ‘Severity hypothesis’, 16g₂;
a likelihood of recovery;
a predicted amount of therapy required, ‘Treatment amount hypothesis’, 16g₃;
a likelihood of the patient not engaging with, or dropping out of, the therapy, ‘Attendance hypothesis’, 16g₄; and/or
a likelihood of the patient benefitting from a particular type of intervention (where multiple intervention options are available).

Some of these outputs 16g are described in more detail in the following sub-sections. —Most Likely Presenting Condition (Diagnosis)—

Preferably, the first stage in a therapy process is to establish a diagnosis, i.e. a hypothesis about a presenting condition.

In face-to-face therapy, the diagnosis is generally based on a conversation between a patient and a therapist during a first session.

In the computer-based system 1, the patient may be asked to complete a self-assessment questionnaire and to provide certain patient data (e.g. personal and medical data). In addition, the patient may be asked to complete specific diagnostic questionnaires, e.g. PHQ-9 (see Kroenke, K., et al. The PHQ-9: validity of a brief depression severity measure. J Gen Intern Med, 16, p. 606, 2001), GAD-7 (see Spitzer, R. L., et al. A Brief Measure for Assessing Generalized Anxiety Disorder: The GAD-7. Arch Intern Med. 166, p. 1092, 2006). This data may be reviewed by the therapist prior to the first therapy session, which may help the diagnosis to be made more quickly and may make better use of the patient-therapist time.

10

Such a process naturally generates data that is preferably stored by the system 1 and may be used as a training set to build a machine learning model for diagnosis. In particular, given (i) a final representation 16c obtained from all of the relevant data relating to the patient (e.g. the text and/or patient data) and (ii) a diagnosis recorded by the therapist, a classification model may be trained using an algorithm from the back-propagation family, such as batch or stochastic gradient descent, Adam, Adagrad, etc.

—Severity—

When diagnosing the presenting condition, the therapist may also make a decision about its severity.

This is normally presented as a numeric value on a scale. For example, in the so-called stepped care model, severity is marked as 1, 2, 3, or 4 (see D. M. Clark, “Implementing NICE guidelines for the psychological treatment of depression and anxiety disorders: the IAPT experience,” International Review of Psychiatry, vol. 23, no. 4, p. 318, 2011).

Again, given (i) a final representation 16c obtained from all of the relevant data relating to the patient and (ii) a severity recorded by the therapist, a regression model may be trained using an algorithm from the back-propagation family.

—Amount of Therapy—

The amount of therapy required, e.g. the number of sessions required, is another numeric value that can be estimated using a regression model in a similar way to severity. —Likelihood of Non-Engagement or Dropping Out—

Patients may not engage with the therapy process, e.g. patients may not present or may give up at the initial stage of therapy process. Patients may also drop out of the therapy process, e.g. by stopping participating after several sessions. Non-presentation, non-participation and/or non-attendance will be understood to mean lack of or reduced adherence to the therapy process on the part of the patient, irrespective of how that process is carried out. For example within the context of internet-enabled or online psychotherapy, therapy delivery may be carried out online, or through a combination of online and face-to-face therapy, or a combination of online and one-to-one therapy over the telephone or other communication means, therefore non-engagement or drop out within that context may mean non-engagement or drop out with/from online therapy, face-to-face therapy and/or one-to-one therapy, or with any combination of the therapy delivery methods.

These occurrences may be modelled as a two-class classification problem, which splits the patients into those who engage (or drop out) and those who do not.

A machine learning classification model may be trained to produce output probabilities that any new patient belongs to one or other of the classes. These probabilities may then be interpreted as the likelihood of engagement (or drop out) for a given patient.

Alternatively, these occurrences (patient non-presentation, or drop-out at an initial or later stage) may be modelled as a regression problem, i.e. one that outputs a numeric regression score. This output may also be referred to as an ‘engagement score’ or an ‘attendance score’.

A machine learning regression model 16d may be trained to produce an output regression score, wherein if the output regression score is high (i.e. a high attendance score) this may be expressed as a low likelihood of non-engagement and/or drop out by the patient, and if the regression score is low (i.e. a low attendance score) this may be expressed as a high likelihood of non-engagement and/or drop out by the patient.

Optionally, a machine learning regression model 16d may be trained to produce an output number wherein the number provides an estimation of the number of sessions a patient will attend.

Test Results 1

The system 1 was tested and found to achieve a correct classification rate (CCR) of 44% in relation to presenting condition. This rate is relative to “ground truth” diagnoses performed by experienced supervisors.

The CCR achieved by the therapists as part of the actual therapy processes was substantially the same, i.e. 44%.

Thus, the results show that the system 1 may be as accurate as therapists in diagnosing presenting condition.

Table 1 below shows a comparison of “ground truth” diagnoses (rows) with predictions made by the system 1 (columns) for nine different presenting conditions. These results were obtained using a development set. With the development set, the system 1 achieves a CCR of about 60% in relation to presenting condition.

TABLE 1

| | | | Predictions | | | | | | | | |
|---|---|---|---|---|---|---|---|---|---|---|---|
| | | | 0 | 1 | 2 | 3 | 4 | 5 | 6 | 7 | 8 |
| Depression | Development set | 0 | 495 | 40 | 0 | 6 | 5 | 2 | 12 | 24 | 0 |
| Generalised_Anxiety_Disorder | | 1 | 75 | 122 | 3 | 1 | 3 | 0 | 16 | 22 | 0 |
| Health_Anxiety | | 2 | 7 | 17 | 13 | 0 | 1 | 0 | 5 | 1 | 0 |
| Long_Term_Conditions | | 3 | 45 | 7 | 1 | 2 | 1 | 0 | 1 | 0 | 0 |
| OCD | | 4 | 17 | 12 | 1 | 0 | 38 | 0 | 4 | 1 | 0 |
| PTSD | | 5 | 29 | 7 | 0 | 1 | 0 | 2 | 3 | 2 | 0 |
| Panic_Disorder | | 6 | 19 | 30 | 2 | 1 | 2 | 0 | 84 | 7 | 0 |
| Social_Anxiety_Disorder | | 7 | 36 | 20 | 0 | 0 | 2 | 0 | 13 | 77 | 0 |
| Specific_Phobia | | 8 | 1 | 4 | 0 | 1 | 1 | 0 | 17 | 2 | 2 |

Fifth Step of the Method

Referring in particular to FIG. 3, at a fifth step S6, one or more actions are taken based on the one or more outputs 16g of the fourth step S4.

As a simple example, an action may involve presenting information relating to one or more outputs 16g to a therapist via the therapist interface.

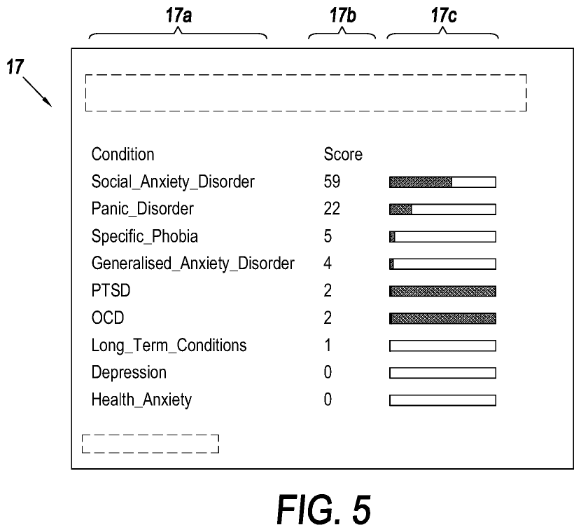

Referring to FIG. 5, the therapist interface may provide a display 17 including a plurality of possible presenting conditions 17a. The presenting conditions are arranged in order of likeliness, with the most likely presenting condition at the top. The display 17 also includes confidence scores 17b for the possible conditions. The display 17 also includes graphical representations 17c of the confidence scores.

This illustrates that the system 1 may be able to predict co-morbidities (when the patient presents with a combination of conditions), i.e. when two or more conditions receive similarly high confidence scores. The system 1 may also be able to provide an indication of when it is unsure about a diagnosis, i.e. when no single presenting condition receives a significantly higher score than the others.

Various other actions may be performed, e.g.:
Allocation of the patient to a therapist;
Estimation of treatment costs;
Suggestion of optimal treatment plans to the therapist;
Deployment of additional interventions to prevent drop-out;
Optimisation of treatment costs by applying the most cost effective intervention likely to lead to a positive outcome (e.g. mild conditions can be treated by less experienced therapists; very mild conditions can be improved through the provision of self-help materials);
Making relevant information and documentation available to the therapist and/or the patient prior to and during the therapy process.

Some actions will be described in more detail in the following sub-sections.

—Allocation of Therapists—

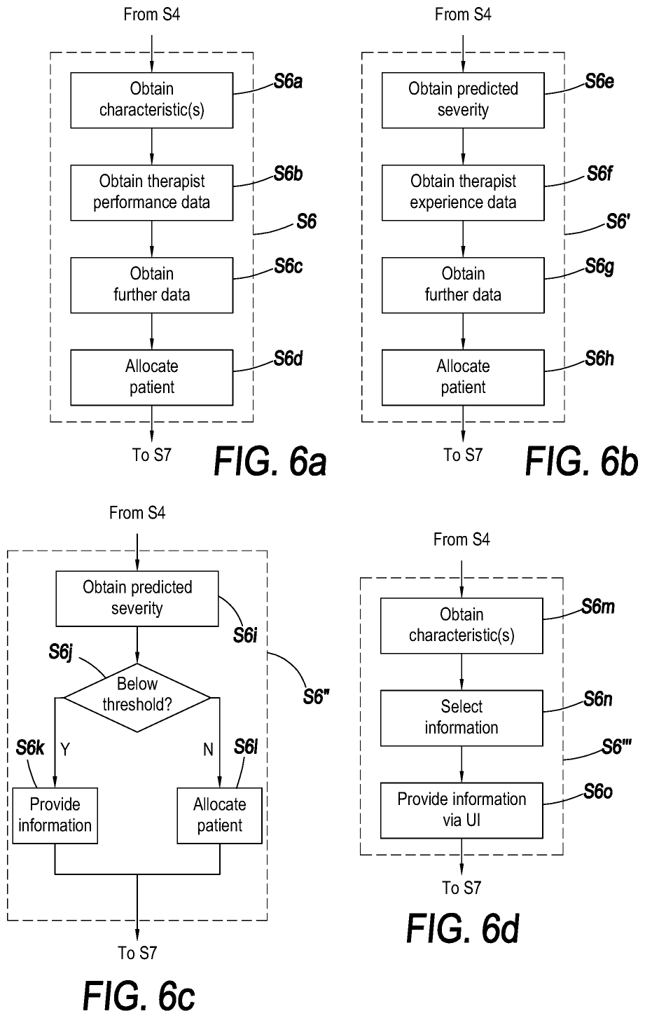

Referring in particular to FIG. 6a, an action S6 may involve the following sub-steps S6a-d, wherein the action relates to the ‘one or more actions based on output’ of FIG. 3, and the action is performed before the end of the method 10.

At a first sub-step S6a, one or more characteristics (hereinafter referred to as relevant characteristics) are obtained. For example, the relevant characteristics may be a most likely presenting condition and a predicted severity of the presenting condition for the patient (hereinafter referred to as the relevant patient).

At a second sub-step S6b, data (hereinafter referred to as relevant therapist performance data) is obtained. The relevant therapist performance data describes performance of each of a plurality of therapists in relation to the relevant characteristics. For example, the relevant therapist performance data may include average outcome measures in relation to patients with the same (or similar) most likely presenting condition and the same (or similar) predicted severity of the presenting condition as the relevant patient. An outcome measure may be of any suitable type, e.g. recovery rate, improvement rate, etc.

At an optional third sub-step S6c, further data relating to the patient and/or to a plurality of therapists is obtained. For example, the further data may relate to availability of the patient and/or the therapists (e.g. dates and times for sessions), workload of the therapists, etc.

At a fourth sub-step S6d, the patient is allocated to one of a plurality of therapists. The allocation is based at least in part on which therapist has the best performance in relation to the characteristics. The allocation may also be based in part on the further data, e.g. relating to availability etc.

Referring in particular to FIG. 6b, another action S6′ may involve the following sub-steps S6e-h.

At a first sub-step S6e, a predicted severity of a presenting condition of the relevant patient is obtained.

At a second sub-step S6f, data describing experience of each of a plurality of therapists (hereinafter referred to therapist experience data) is obtained. The therapist experience data may (or may not) be specific to the most likely presenting condition of the relevant patient.

At an optional third sub-step S6g, further data may be obtained in the same way as the sub-step S6c (FIG. 6a).

At a fourth sub-step S6h, the patient is allocated to one of a plurality of therapists. Patients with more severe conditions are allocated to therapists with more experience. This may be done in any suitable way. The allocation may also be based on the further data, e.g. relating to availability etc.

—Avoiding Unnecessary Use of Therapists—

Referring in particular to FIG. 6c, another action S6″ may involve the following sub-steps S6i-l.

At a first sub-step S6i, a predicted severity of a presenting condition of the patient is obtained. This is the same as sub-step S6e (FIG. 6b).

At a second sub-step S6j, it is determined whether or not the severity is equal to or below a predetermined threshold. This severity threshold is determined in any suitable way so as to separate very mild conditions that may not (immediately) require a therapist from less mild conditions that do. In order to determine the severity threshold, data from a cohort of patients of known outcome (e.g. severity) may be used to set the threshold; the threshold may then be applied to a matched cohort of new patients.

If it is determined that the severity is equal to or below the threshold, then the method 10 proceeds to a third sub-step S6k. At the third sub-step S6k, the system 1 initiates a therapy process that does not directly (or indirectly) involve a therapist. This process preferably involves providing information to the patient via the system 1 (see next sub-section).

If it is determined that the severity is above the threshold, then the method 10 proceeds to a fourth sub-step S6l. At the fourth sub-step S6l, the patient is allocated to a therapist. This may be performed as described above in relation to FIG. 6a or 6b.

Furthermore, more than one severity threshold may be determined as appropriate. For example, a clinician (therapist) may define a plurality of tiers of severity (i.e. more than two tiers), and accompanying best-practice recommendations associated with the treatment of patients in each severity tier. The (severity) threshold(s) of the method may then be determined such that the method allocates patients to a particular severity tier with a high likelihood of correct allocation. By way of further example, the severity thresholds of the method may be determined such that they separate patients into the IAPT-defined severity classes denominated steps 2, 3 and 4. In order to determine the severity threshold(s), data from a cohort of patients of known outcome (e.g. severity) may be used to set the threshold(s); the threshold(s) may then be applied to a matched cohort of new patients.

—Presenting Relevant Information—

Referring in particular to FIG. 6d, another action S6′″ may involve the following sub-steps S6m-o.

At a first sub-step S6m, one or more characteristics (hereinafter referred to as relevant characteristics) are obtained. This is the same as sub-step S6a (FIG. 6a).

At a second sub-step S6n, a subset of a set of information is selected based on the relevant characteristics. For example, information relating to a most likely presenting condition may be selected, etc.

At a third sub-step S6o, the selected information is provided to the therapist and/or to the patient via a user interface of the system 1. The information may include documents, questionnaires, etc. The information may be provided at appropriate times, e.g. before or during particular sessions. Thus, the method may help the therapist and the patient during the therapy process.

—Deployment of Additional Interventions to Prevent Drop-Out—

Another action may involve the following: an attendance score for a patient (a regression output score; inversely related to a predicted likelihood of the relevant patient not engaging or dropping out) is obtained.

One or more attendance score threshold values (T1, T2, etc) are pre-determined. The threshold(s) are determined in any suitable way so as to provide a meaningful separation of different likelihoods of non-engagement or drop-out by a patient. The (attendance score) threshold(s) may be adjusted to balance the risks of false positives and false negatives. For different levels of control, more or fewer thresholds may be defined as desired. In order to determine the attendance score threshold(s), data from a cohort of patients of known outcome (e.g. likelihood of drop-out) may be used to set the threshold(s); the threshold(s) may then be applied to a matched cohort of new patients.

Then it may be determined whether the attendance score for the patient is above or below the one or more pre-determined (attendance score) thresholds. For example, where two thresholds (T1 and T2) are used, it is determined if the attendance score for the patient is equal to or below T1, between T1 and T2, or equal to or above T2.

The method may then proceed to a sub-step wherein the patient is allocated to a category of risk of non-engagement and/or drop-out (risk category). In the non-limiting example above where two attendance score thresholds are used: if the attendance score for a patient is equal to or below T1 the patient is allocated to the category ‘high risk’; if the attendance score is between T1 and T2 the patient is allocated to the category ‘medium risk’; if the attendance score is equal to or above T2 the patient is allocated to the category ‘low risk’. Allocation of a patient to a particular category of risk of non-engagement and/or drop-out may be considered to mean the likelihood of non-engagement and/or drop-out by that patient meets a predetermined criterion.

In a subsequent sub-step, one or more interventions may be deployed in response to the risk category to which the patient is allocated. The risk category may be made available to the clinical team who manage the patient's therapy, who may then deploy one or more intervention(s). Suitable interventions would be predicted or known to decrease the likelihood of patient non-engagement or drop out (i.e. to increase therapy engagement or attendance). Such interventions may include, but are not limited to:
i. booking blocks of multiple sessions at the same time, rather than one session at a time;
ii. contacting or calling the patients in between sessions to e.g. reinforce the importance of the therapy process for their recovery;
iii. explaining to the patient e.g. what progress they are expected to make, and/or how many sessions are normally needed to help someone like them.

Alternatively, the risk category may be used by the system to allocate the patient to a particular intervention(s); the deployment of the intervention(s) may subsequently be carried out by the clinical team.

Intervention(s) may be deployed to those patients allocated to one of the categories, for example the high risk category. Alternatively, intervention(s) may be deployed to patients in multiple categories, for example to those patients who are allocated to either the high or the medium risk category. Another way of expressing this is that intervention(s) may be deployed to patients in categories other than the lowest risk category. Furthermore, different intervention(s) may optionally be deployed depending on risk category, for example more interventions may be deployed to high risk patients than to medium risk patients, or interventions known or predicted to be more effective may be selected for high risk patients. The decision to deploy a particular intervention, or a plurality of interventions, to a particular risk category of patients may be based on obtaining a balance between the cost of the intervention(s) (e.g. monetary cost) and the cost of drop-out (patient does not complete treatment and therefore does not improve/recover). Alternatively or additionally, the attendance score threshold values which define the risk categories into which the patients are allocated may be set to balance the cost of intervention with the cost of drop-out.

An alternative way of expressing this is that the deployment of intervention(s) depends on or is in response to the likelihood of non-engagement and/or drop-out by the patient meeting a predetermined criterion, wherein the predetermined criterion may be, for example, allocation to a risk category above the lowest risk category.

It is advantageous to obtain an output of the method (for example an attendance score for a patient; or optionally the resultant allocation of a patient to a category of risk of non-engagement and/or drop-out) at the start of the therapy process or before the therapy process begins, because intervention(s) to increase engagement may consequently only be deployed to those patients at higher risk. This may therefore reduce the overall cost of providing intervention(s), whilst at the same time achieving a reduction in non-engagement and/or drop out occurrence amongst patients. It is advantageous to be able to predict which patients are at higher risk of non-engagement and/or drop out and to deploy intervention(s) before non-engagement and/or drop out occurs, rather than reacting to drop-out after it has happened; intervention(s) deployed in advance of drop out may be more effective in increasing engagement.

The choice of attendance score threshold(s) reflects decisions with regard to the cost of the additional interventions and the cost of the patient dropping-out. Each possible threshold value corresponds to a given probability of false positives (identifying a patient who would not have dropped out as being at risk) and false negatives (missing a patient who will end up dropping out). Increasing a threshold makes the model more sensitive, reducing false negatives, but increasing false positives. Lowering a threshold makes the model less sensitive, increasing false negatives, but decreasing false positives. The chosen attendance score threshold corresponds to a given balance between these two types of error.

The thresholds are chosen to optimise the benefit to the patient, given a constraint on the maximum acceptable cost.

Optionally, the non-engagement and/or drop-out risk category, and one or more other output(s) predicting a characteristic of a condition of the patient and/or of the therapy process, may be obtained for a particular patient. For example, the non-engagement and/or drop-out risk category, and the predicted amount of therapy required, may both be obtained for a particular patient. By way of further example, the non-engagement and/or drop-out risk category, and the predicted severity of a condition at the initial stage, may both be obtained for a particular patient. These multiple outputs may be used in combination, or synergistically, to make a decision about the deployment of one or more intervention(s).

For example, where the amount of therapy required is estimated to be high and the non-engagement and/or drop-out risk category is also ‘high’, the decision may be taken to deploy multiple interventions, or interventions known to have a greater positive effect on patient engagement.

Alternatively, one or more of the outputs, for example the predicted amount of therapy required, may be used in the determination of the threshold(s) used to assess the attendance score.

Further Method

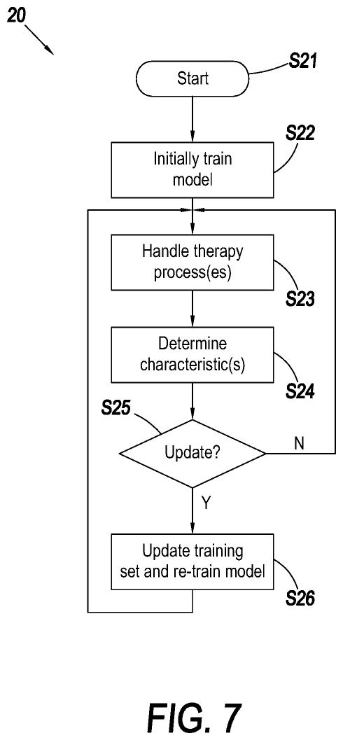

Referring to FIG. 7, the system 1 may perform a further method 20 comprising several steps S21-S26.

At a first step S21, the method 20 starts.

At a second step S22, the deep learning model 16d is initially trained. This is performed using an initial training set that preferably includes data relating to a plurality of (past) patients. The (initial) training is performed as described above.

At a third step S23, one or more therapy processes are handled by the system 1. For each therapy process, the abovedescribed method 10 will be performed. Thus, among other things, text 16a and patient data 16b may be obtained by the system 1. Furthermore, the patient and therapist (if a therapist has been allocated) may exchange text-based messages during several sessions of therapy. All relevant data relating to these activities is preferably stored by the system 1.

At a fourth step S24, one or more characteristics of a condition of a patient and/or of a therapy process are determined. Determining a characteristic may involve extracting relevant data relating to an ongoing therapy process. For example, the presenting condition and/or its severity may be determined by a therapist and/or a supervisor based certain data. The system 1 may prompt this. The amount of therapy required, non-engagement, etc. may be determined by the system 1 based on records of therapy sessions, etc.

At a fifth step S25, it is determined whether or not to update the training. Updating of the training may carried out periodically or in response to one or more particular criteria being met. For example, instances where a predicted characteristic is subsequently determined to be incorrect may be particularly significant. If it is determined that the training is to be updated, then the method 20 proceeds to a sixth step S26; otherwise, the method 20 returns to the third step S23.

At the sixth step S26, the training set is updated using data obtained at the third and fourth steps S23, S24, and then the deep learning model 16d is re-trained using the updated training set. The (re-)training is performed as described above.

The method 20 then returns to the third step S23.

Other Modifications

It will be appreciated that many other modifications may be made to the abovedescribed embodiments.

For example, the methods 10, 20 may be used in relation to similar text 16a to take actions S4a in applications other than computer-based systems 1 for providing psychotherapy. Other applications may include, for instance, systems for monitoring wellbeing.

Therapist assistance by the system 1 may be extended, for example, to support protocol adherence, which is important in achieving good recovery and improvement measures (see A. Gyani, R. Shafran, R. Layard and D. M. Clark, “Enhancing recovery rates: lessons from year one of IAPT,” Behaviour Research and Therapy, vol. 51, no. 9, p. 597, 2013.). To achieve this, the system 1 may: provide links to key points before each therapy session begins, and monitor each therapy session; and provide just-in-time reminders and prompts. The system 1 may also identify correlations between actions and outcomes, etc.

Test Results 2

The system 1 was subsequently tested using a second dataset of ground truth cases. The ground truth dataset used was a random selection of real cases incoming to the therapy service. The number of cases included in the dataset increased over time. The cases in the dataset were manually tagged by a team of 3 clinical supervisors. These were highly experienced clinicians, who provide reliable diagnoses for the cases.

Using this second dataset, the Correct Classification Rate (CCR) scores for the human therapists and for the triage AI system changed over time as shown in Table 2 below. This demonstrates that the triage AI system has improved over time, and has reached the same level of accuracy as the cohort of human therapists. That means that the AI triage system has demonstrated diagnosis accuracy equivalent to the average therapist; the AI triage system may have projected diagnosis accuracy greater than that of the average therapist. The main driver of these improvements is the increase with time of the number of cases that can be used to train the machine learning models, as well as, to a lesser degree, fine tuning the configuration of the training process.

TABLE 2

| | |
|---|---|
| No. tagged | Correct Classification Rate |

| Time | cases | Therapists | Triage AI |
|---|---|---|---|
| Month 1 | 166 | 51.56% | 46.99% |
| Month 2 | 215 | 54.04% | 50.23% |
| Month 6 | 215 | 54.04% | 54.88% |

Thus, the results show that the system 1 may be as accurate as human therapists in diagnosing presenting conditions. Extrapolation of the results shows that the system 1 may have greater accuracy in diagnosing presenting conditions than human therapists. Table 3 below shows a comparison of “ground truth” diagnoses (rows) with predictions made by the system 1 (columns) for ten different presenting conditions. These results were obtained using the second dataset as per the CCR scores presented in Table 2, where the “ground truth” diagnoses were those for the 215 cases manually tagged by a team of 3 experienced clinical supervisors.

The number of cases in each row of Table 3 reflects the prevalence of each of the corresponding conditions in the patient population. As can be expected, the AI system performs worse on conditions with low prevalence, such as OCD, or PTSD, for which a smaller number of training examples were available. This effect is less present for conditions associated with very specific language, such as social anxiety, where the system performs well despite the smaller number of examples.

The improvement in CCR scores illustrated by Table 2 is expected to continue over time of use of the AI triage system, so the accuracy on all conditions and overall will improve as the training dataset increases.

TABLE 3

| | | AI Triage Predictions | | | | | | | | | | |
|---|---|---|---|---|---|---|---|---|---|---|---|---|
| | | | | | Generalised | | | | | | Social | |
| | | | Other | Depres- | Anxiety | Health | Long Term | | | Panic | Anxiety | Specific |
| | | Agoraphobia | conditions | sion | Disorder | Anxiety | Conditions | OCD | PTSD | Disorder | Disorder | Phobia |
| Ground | Agoraphobia | 0 | 0 | 1 | 1 | 0 | 0 | 0 | 0 | 0 | 0 | 0 |
| Truth | Other | 0 | 0 | 26 | 7 | 0 | 0 | 1 | 0 | 0 | 0 | 0 |
| | conditions | | | | | | | | | | | |
| | Depression | 0 | 0 | 81 | 13 | 0 | 0 | 0 | 0 | 3 | 1 | 0 |
| | Generalised | 0 | 0 | 10 | 19 | 1 | 0 | 0 | 0 | 2 | 1 | 0 |
| | Anxiety | | | | | | | | | | | |
| | Disorder | | | | | | | | | | | |
| | Health | 0 | 0 | 1 | 5 | 0 | 0 | 0 | 0 | 1 | 0 | 0 |
| | Anxiety | | | | | | | | | | | |
| | Long Term | 0 | 0 | 2 | 4 | 0 | 0 | 0 | 0 | 0 | 0 | 0 |
| | Conditions | | | | | | | | | | | |
| | OCD | 0 | 0 | 2 | 1 | 0 | 0 | 0 | 0 | 0 | 0 | 0 |
| | PTSD | 0 | 0 | 4 | 1 | 0 | 0 | 0 | 0 | 0 | 0 | 0 |
| | Panic Disorder | 0 | 0 | 3 | 1 | 0 | 0 | 0 | 0 | 7 | 0 | 0 |
| | Social Anxiety | 0 | 0 | 1 | 0 | 0 | 0 | 0 | 0 | 0 | 8 | 0 |
| | Disorder | | | | | | | | | | | |
| | Specific | 0 | 0 | 3 | 1 | 0 | 0 | 0 | 0 | 0 | 0 | 3 |
| | Phobia | | | | | | | | | | | |

Test Results 3

The performance of the system in relation to the prediction of drop-out (drop-out indicator) was evaluated. A machine learning regression model was trained to output a number (a regression output score, e.g. an attendance score) for a particular patient, based on the available text data, and optionally patient data (further data), inputs.

The model was trained using a development dataset. In this model, a higher regression output score indicates a higher likelihood of patient engagement (a lower likelihood of patient drop out).

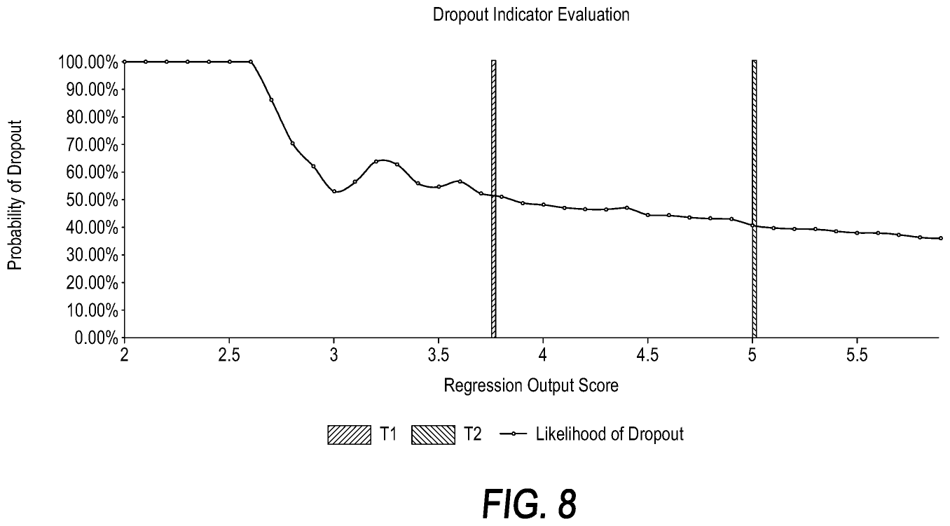

The regression output scores produced by the trained model for the dataset were plotted against the actual drop-out data collected for the dataset (probability of dropout), see FIG. 8 and Table 4. From FIG. 8 and Table 4 it can be seen that for this dataset all patients for whom the model produced a regression output score of 2.6 or less were 100% likely to drop out. Two thresholds (T1 and T2) were defined for the evaluation. T1 was set at a regression output score of 3.75 (corresponding to approximately 50% probability of drop out); T2 was set at a regression output score of 5.00 (corresponding to approximately 40% probability of drop out). These thresholds may be considered examples of attendance score thresholds.

Patients for whom the model estimated a regression output score equal to or less than T1 were considered ‘high risk of drop-out’; patients for whom the model estimated a regression output score between T1 and T2 were considered as ‘medium risk’; patients for whom the model estimated a regression output score of equal to or greater than T2 were considered as low risk' of drop out. The proportion of patients allocated to the ‘high risk of drop-out’ category was 10.3%, the proportion of patients allocated to the ‘medium risk of drop-out’ category was 48.9%, and the proportion of patients allocated to the low risk of drop-out' category was 40.8%.

Therefore if one or more interventions predicted or known to increase engagement are deployed to patients only in the high risk and medium risk categories, this represents a 40.8% cost saving compared with uniformly treating the entire group. Given the actual drop-out rate measured for IECBT therapy (34%), deploying interventions with only patients allocated to the high risk and medium risk categories using these or similar thresholds will effectively target those patients most likely to drop-out without unnecessary use of resources. The threshold(s) of the method may be determined in such a way as to balance the cost of deploying intervention(s) with the cost of non-engagement/drop-out.

TABLE 4

| Regression | Likelihood of |
|---|---|
| output score | drop out |
| 1.5 | 100.00% |
| 1.6 | 100.00% |
| 1.7 | 100.00% |
| 1.8 | 100.00% |
| 1.9 | 100.00% |
| 2.0 | 100.00% |
| 2.1 | 100.00% |
| 2.2 | 100.00% |
| 2.3 | 100.00% |
| 2.4 | 100.00% |
| 2.5 | 100.00% |
| 2.6 | 100.00% |
| 2.7 | 85.71% |
| 2.8 | 70.00% |
| 2.9 | 61.54% |
| 3.0 | 52.63% |
| 3.1 | 56.00% |
| 3.2 | 63.33% |
| 3.3 | 62.16% |
| 3.4 | 55.32% |
| 3.5 | 54.10% |
| 3.6 | 56.00% |
| 3.7 | 51.65% |
| 3.8 | 50.47% |
| 3.9 | 48.25% |
| 4.0 | 47.49% |
| 4.1 | 46.51% |
| 4.2 | 45.93% |
| 4.3 | 45.82% |
| 4.4 | 46.28% |
| 4.5 | 43.85% |
| 4.6 | 43.69% |
| 4.7 | 42.86% |
| 4.8 | 42.50% |
| 4.9 | 42.27% |
| 5.0 | 40.07% |
| 5.1 | 39.14% |
| 5.2 | 38.78% |
| 5.3 | 38.71% |
| 5.4 | 37.85% |
| 5.5 | 37.29% |
| 5.6 | 37.18% |
| 5.7 | 36.56% |
| 5.8 | 35.63% |
| 5.9 | 35.24% |

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A method for use by a computer-based system for providing psychotherapy, the method comprising:
obtaining, via a user interface of the system, text data relating to a patient at an initial stage of a therapy process;
using at least a first part of a deep learning model to obtain a representation of at least the text data;
using at least a second part of the deep learning model, and an input thereto formed using the representation, to obtain an output predicting a characteristic of a condition of the patient and/or of the therapy process; and
causing the system to take one or more actions relating to the therapy process, wherein the one or more actions are selected based on the output;
wherein the deep learning model is trained using a training set comprising, for each of a plurality of other patients, text data relating to the other patients at an initial stage of a therapy process and a result of a determination of the characteristic.

2. A method according to claim 1, wherein the text data comprises free-form text.

3. A method according to claim 1, comprising:
obtaining further data relating to the patient; and
obtaining the representation by at least:
obtaining an intermediate representation of the text data;
obtaining a further intermediate representation of the further data; and
joining the intermediate representations.

4. A method according to claim 1, wherein obtaining the representation comprises pre-processing an intermediate representation of at least the text data, wherein the pre-processing comprises, normalising.

5. A method according to claim 1, wherein the first part of the deep learning model is pre-trained using in-domain text.

6. A method according to claim 1, where the second part of the deep learning model performs classification or regression.

7. A method according claim 1, wherein the second part of the deep learning model performs a plurality of instances of classification and/or regression to obtain a plurality of outputs.

8. A method according to claim 1, wherein the output is selected from the group consisting of:
a most likely condition at the initial stage;
a likelihood score for each of a set of possible conditions at the initial stage;
a predicted severity of a condition at the initial stage;
a predicted amount of therapy required;
a likelihood of non-engagement and/or drop-out by the patient;
one of a plurality of therapy options that is most likely to be beneficial; or,
a combination thereof.

9. A method according to claim 1, wherein the one or more actions comprise allocating the patient to one of a plurality of therapists, wherein the action is selected from the group consisting of:
the allocation is based on a predicted characteristic and on data describing performance of the therapist in relation to the predicted characteristic; t
the allocation is based on a predicted severity of a condition at the initial stage and on data describing experience of the therapist, wherein patients with more severe conditions are allocated to therapists with more experience; or,
a combination thereof.

10. A method according to claim 1, wherein the one or more actions comprise selecting at least one of a plurality of therapy plans based on the output and providing, via a user interface of the system, an indication of the selected at least one therapy plan to the therapist.

11. A method according to claim 10, wherein the one or more actions comprise, in response to the likelihood of non-engagement or drop-out by the patient meeting a predetermined criterion, deploying at least one of a plurality of interventions, wherein the at least one intervention is predicted or known to increase engagement.

12. A method according to claim 11, wherein the one or more actions comprise, in response to a predicted severity of a condition being below a predetermined criterion or threshold, initiating a therapy process that comprises providing information to the patient via the system.

13. A method according to claim 1, comprising selecting a subset of a set of information based on the output and providing, via a user interface of the system, the selected information to the therapist and/or to the patient.

14. A method according to claim 1, comprising:
subsequently determining the characteristic of a condition of the patient, the therapy process, or both; and
selectively updating the training set, re-training the deep learning model, or both.

15. (canceled)

16. (canceled)

17. (canceled)

18. A method comprising:
vectorising a first text data relating to a patient at an initial stage of a therapy process to produce a plurality of first text data tensors;
extracting a plurality of features that represent the first text data from the plurality of first text data tensors using a first portion of a deep learning model;
analysing a representation, based on at least the plurality of features that represent the first text data, with a classification/regression portion of the deep learning model, thereby producing an output correlated to at least one characteristic of a condition of the patient, a related therapy process, or both; and
categorising the patient based on the output;
wherein the deep learning model is trained using at least second text data from other patients at an initial stage of a therapy process and a corresponding characteristic of a condition of the other patients.

19. The method of claim 18 further comprising:
vectorising patient data relating to the patient to produce a plurality of patient data tensors;
wherein the representation analysed by the classification/regression portion of the deep learning model is further based on the plurality of patient data tensors.

20. The method of claim 18, wherein the deep learning model is further trained using in-domain text.

21. The method of claim 18, wherein the classification/regression portion of the deep learning model performs a classification process on the representation; and
wherein the at least one characteristic of a condition of the patient, the related therapy process, or both comprises a most likely condition of the patient at the initial stage and a likelihood score for each of a set of possible conditions at the initial stage.

22. (canceled)

23. The method of claim 18, wherein the classification/regression portion of the deep learning model performs a regression process on the representation; and wherein the at least one characteristic of a condition of the patient and/or the related therapy process comprises a predicted severity of the condition at the initial stage.

24. The method of claim 23, wherein
when the predicted severity of the condition at the initial stage is below a threshold, categorising the patient based on the output comprises initiating a therapy process that initially does not directly involve a therapist; and,
when the predicted severity of the condition at the initial stage is above a threshold, categorising the patient based on the output comprises initiating a therapy process with an experienced therapist.

25. (canceled)

26. The method of claim 18, wherein the classification/regression portion of the deep learning model performs a regression process on the representation; and
wherein the output is correlated to at least one characteristic of a condition of the patient, the related therapy process, or both comprises a predicted amount of therapy required and one of a plurality of therapy options that is most likely to be beneficial.

27. The method of claim 18, wherein the classification/regression portion of the deep learning model performs a regression process on the representation; and
wherein the output correlated to at least one characteristic of a condition of the patient, the related therapy process, or both comprises a likelihood of non-engagement or drop-out by the patient, and
wherein categorising the patient comprises deploying at least one of a plurality of interventions, wherein the at least one intervention is predicted or known to increase engagement.

28. (canceled)
